(19) United States
(12) Patent Application Publication
(10) Pub. No.: US 2024/0407647 A1
(43) Pub. Date: Dec. 12, 2024

(54) CONSTANT DOWNFORCE ASSEMBLY FOR CONTACT LENS-BASED WIDE ANGLE VISUALIZATION

(71) Applicant: Alcon Inc., Fribourg (CH)

(72) Inventor: Steven T. Charles, Memphis, TN (US)

(73) Assignee: Alcon Inc., Fribourg (CH)

(21) Appl. No.: 18/667,000

(22) Filed: May 17, 2024

Related U.S. Application Data
(60) Provisional application No. 63/506,713, filed on Jun. 7, 2023.

(51) Int. Cl. A61B 3/13; A61B 3/00; A61B 3/14

(52) U.S. Cl. CPC A61B 3/13; A61B 3/0041; A61B 3/145

(57) ABSTRACT

A contact-based wide-angle visualization system (“WAVS”) for use in an ophthalmic suite having a microscope includes a constant downforce (CDF) assembly and a contact lens device. The CDF assembly has a proximal end and a distal end. The proximal end connects to an optical head of the microscope via an intervening connecting arm. The contact lens device connects to the distal end of the CDF assembly and includes a contact lens configured to be worn on a cornea of a patient's eye in the ophthalmic suite. The CDF assembly provides a constant downforce to the contact lens device, and self-levels and thereby maintains the contact lens device in an approximately parallel orientation relative to the floor of the ophthalmic suite.

### CROSS-REFERENCE TO RELATED APPLICATIONS

The present application claims the benefit of priority to U.S. Provisional Application No. 63/506,713 filed Jun. 7, 2023, which is hereby incorporated by reference in its entirety.

### INTRODUCTION

The present disclosure relates to wide angle visualization of the inner anatomy of a patient's eye. As appreciated by those skilled in the art, the proper prognosis and diagnosis of retinal tears and other intraocular conditions often requires a physician to employ a high-definition optical system. Such a system magnifies the eye via a microscope to facilitate visualization, with image capture via a digital camera also being possible as needed. In this manner, the physician is afforded a clear real-time view of the retina, macula, vitreous body, and surrounding tissues within the eye.

During an ophthalmic visualization procedure, a physician might require a wider view of the patient's vitreous chamber than is ordinarily achievable using the microscope's lenses and visualization hardware. For instance, the physician may find it beneficial to view the peripheral retina area when monitoring for conditions such as retinal tears or detachments. Wide-angle visualization can be performed using a specially-constructed optical lens, which in some implementations is placed directly on the patient's cornea (“contact-based”). In other implementations the lens remains a short distance away from the cornea (“non-contact-based”). The shape and construction of the lens in either case provides the desired wide-angle view assisted by endoillumination.

### SUMMARY

Disclosed herein is a contact-based wide-angle visualization system (“WAVS”) for use in an ophthalmic suite equipped with a microscope. The contact-based WAVS as contemplated herein includes a constant downforce (CDF) assembly having oppositely-disposed proximal and distal ends. The proximal end of the CDF assembly is connectable to the microscope via an intervening connecting arm, e.g., an articulatable or translatable arm or arms as set forth herein. A contact lens device is connectable to the distal end of the CDF assembly, with the contact lens device having a contact lens configured to be worn on a cornea of a patient's eye in the ophthalmic suite. The CDF assembly provides a predetermined/calibrated constant downforce to the contact lens device and its contact lens at a level sufficient for retaining the contact lens on the cornea without corneal distortion. Additionally, the CDF assembly is configured to self-level and thereby maintain the contact lens device in an approximately parallel orientation relative to a floor of the ophthalmic suite, e.g., within about ±5° to 10° of true parallel.

In one or more embodiments, a gimbal is connected to the distal end of the CDF assembly and to the contact lens device to thereby maintain the above-noted approximately parallel orientation of the contact lens, for instance by limiting the pitch and/or roll of the contact lens device, with limited tip/tilt resulting in a better view of the critical peripheral retina.

The contact lens device in one or more embodiments includes a support frame and a support frame arm. The support frame in this configuration is configured to support the contact lens. The support frame arm is connected to the support frame and to the CDF assembly. The CDF assembly in this particular non-limiting exemplary configuration includes a shaft circumscribed by a bearing housing containing instrument bearings therein, with the bearing housing being translatable along a longitudinal axis of the shaft, e.g., in response to a force from the contact lens device due to the patient's movements.

One or more constant force springs could be connected to or surround the shaft in a possible implementation. In other implementations, the CDF assembly includes a miniature gas spring.

In an alternative configuration, a four-bar mechanism may be operatively connected to a low-friction air cylinder having a piston disposed therewithin. To ensure an unobstructed view of the ocular anatomy, a longitudinal axis of the piston in such an embodiment could be laterally offset from the four-bar mechanism by a short distance, e.g., using a short interconnecting piece. An end of the piston could be operatively connected to the above-summarized contact lens device.

The low-friction air cylinder in accordance with a non-limiting exemplary embodiment is constructed from glass, e.g., borosilicate glass, or from another application-suitable low-friction material, with a low-friction piston movable within the air cylinder.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 is a schematic illustration of an exemplary ophthalmic suite equipped with a contact-based wide-angle visualization system (“WAVS”) as set forth herein.

FIGS. 2A and 2B illustrate the contact-based WAVS when used with an articulatable connecting arm and a translatable connecting arm, respectively.

FIG. 3 illustrates the contact-based WAVS of FIGS. 1-2B in accordance with a possible construction incorporating linear bearings and constant-force springs.

FIG. 4 depicts the contact-based WAVS of FIGS. 1-3 in accordance with an alternative embodiment incorporating a gas cylinder.

FIG. 5 is an illustration of the contact-based WAVS of FIGS. 1-4 in accordance with a possible construction incorporating a four-bar mechanism and the gas cylinder of FIG. 4.

The appended drawings are not necessarily to scale, and may present a somewhat simplified representation of various features of the present disclosure as disclosed herein, including, for example, specific dimensions, orientations, locations, and shapes. Details associated with such features will be determined in part by the particular intended application and use environment.

### DETAILED DESCRIPTION

Embodiments of the present disclosure are described herein. It is to be understood, however, that the disclosed embodiments are merely examples, and that other embodiments can take various and alternative forms. The Figures are not necessarily to scale. Some features may be exaggerated or minimized to show details of particular components. Therefore, specific structural and functional details disclosed herein are not to be interpreted as limiting, but merely as a representative basis for teaching one skilled in the art to variously employ the present disclosure.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference, which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

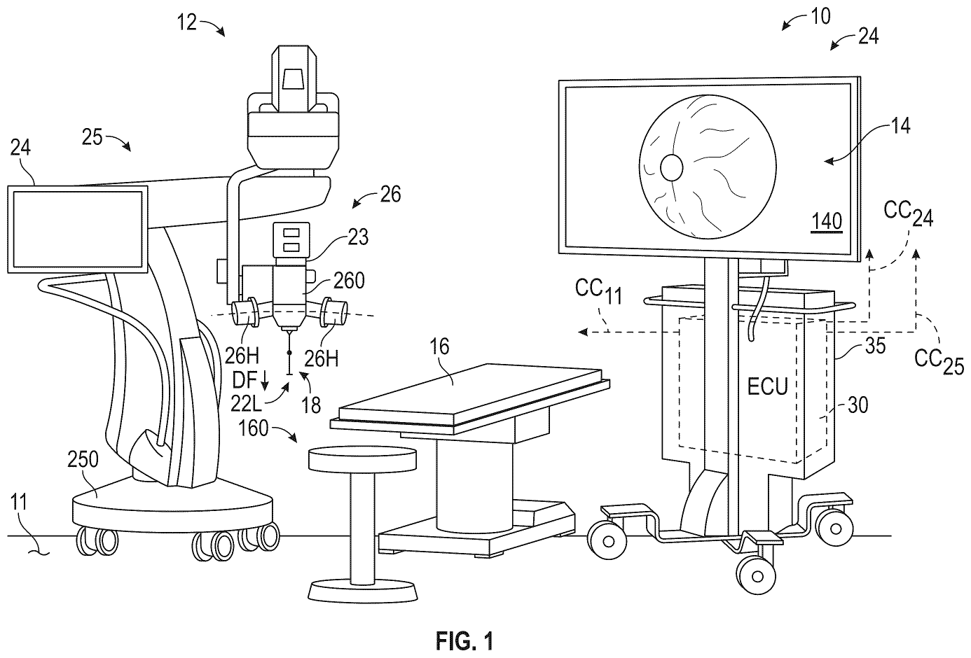

Referring to the drawings, wherein like reference numbers refer to like components, a representative ophthalmic suite 10 is shown schematically in FIG. 1. The ophthalmic suite 10 includes an optical system 12 operable for visualizing intraocular anatomy 14 of a patient's eye 140, a portion of which is projected onto a high-resolution monitor 24 in FIG. 1. While the surgeon and patient are both omitted from FIG. 1 for illustrative simplicity, those skilled in the art will appreciate that the patient would be situated on a platform 16, e.g., a table or reclined in a chair, with the surgeon seated on a stool 160 adjacent to the platform 16. The surgeon would then electronically view the patient's eye 140 in a “heads up” manner with magnification provided by the optical system 12, e.g., via the commercially-available NGENUITY® 3D Visualization System from Alcon, Inc.

In accordance with the present disclosure, the optical system 12 as contemplated herein includes a contact-based wide-angle visualization system (“WAVS”) 17. Although non-contact approaches to wide-angle viewing remain prevalent in the art, it is recognized herein that non-contact alternatives can be challenging to properly implement. For example, non-contact-based WAVS alternatives involve the use of lenses that are connected to an optical head 260 of an ophthalmic microscope 26 rather than worn on the patient's eye 140. As a result, non-contact alternatives for wide-angle viewing are highly sensitive to the patient's movements, requiring almost constant xy-plane translational corrections of the microscope 26. The required corrections are typically driven by the physician's foot pedal inputs, with the physician's movements in turn possibly exacerbating the patient's movements. Also, for proper wide-angle viewing the external/non-contact lens must be placed in close proximity to the cornea. This results in frequent bumping of the cornea surface by the external lens, and with it, a transfer of viscoelastic material from the cornea to the lens, thus necessitating frequent cleaning of the lens.

In contrast, use of a contact-based approach to wide-angle viewing eliminates corneal asphericity, e.g., from prior radial keratotomy, astigmatic keratectomy, or penetrating keratoplasty surgery, or from corneal laceration and other factors. At the same time, a contact-based approach provides an approximately 10° increased field-of-view relative to competing non-contact techniques. However, current contact-based wide-angle viewing alternatives face unique challenges of their own, including difficulty in placing and maintaining the contact lens on the cornea by a surgical assistant due to factors such as patient head or eye movements. This is true regardless of whether such movements are lower in amplitude and repetitive, e.g., due to normal respiration, or more sudden and unexpected, such as large amplitude head movements when the patient suddenly wakes up or jerks, e.g., due to sleep apnea, or sneezes, coughs, or otherwise experiences a startle reflex. The improvements described in detail hereinbelow are therefore intended to address these and other safety issues and other potential problems commonly associated with contact-based wide-angle viewing.

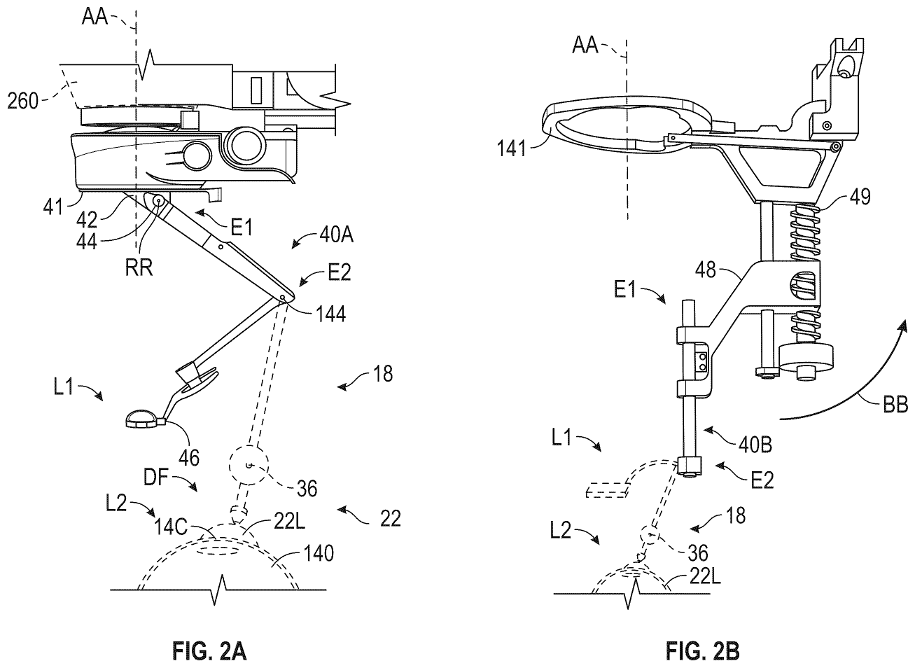

As appreciated in the art and described in detail below with references to the Figures, the contact-based WAVS 17 is constructed such that a constant and balanced downforce (arrow DF) is provided to a contact lens 22L when the contact lens 22L is worn on the patient's eye 140. At the same time, the constant downforce prevents formation of air bubbles or air pockets beneath the contact lens 22L. Thus, potential problems of corneal distortion from too high of a downforce and entrapped air bubbles from an insufficient downforce are alleviated by the construction of the contemplated contact-based WAVS 17 of FIG. 1, exemplary embodiments of a constant downforce (CDF) assembly 18 of which are described below with reference to FIGS. 2A-5.

The optical system 12 illustrated in FIG. 1 includes the microscope 26, e.g., a digital or analog medical-grade microscope device having handles 26H and eye pieces or oculars (not shown). In some embodiments, the microscope 26 could be connected to a digital camera 23 to allow the physician or attending staff to take digital pixel images of the eye 140 as needed. Visualization of the eye 140 may be enhanced by real-time video broadcasting via one or more of the monitors 24, such as a medical grade 4K or other ultra-high definition organic light-emitting diode (OLED) panel, which is situated within easy view of the surgeon and other attending personnel within the ophthalmic suite 10.

As part of the present approach, the optical system 12 shown in FIG. 1 is configured for magnifying and clearly visualizing the intraocular anatomy 14 of the eye 140 in real-time. To that end, the microscope 26 may be suspended from overhead, e.g., connected to and/or supported by a multi-axis robot arm 25. The contact-based WAVS 17 as described herein may be directly or indirectly attached to the optical head 260 of the microscope 26 using a mechanical engagement element, such as the intervening connecting arms 40A or 40B respectively depicted in FIGS. 2A and 2B. Non-limiting exemplary microscopes 26 include the LuxOR® Revalia™ Ophthalmic Microscope from Alcon, Inc., as well as the OPMI Lumera® 700 from Carl Zeiss Meditec, Inc. Other commercially-available microscopes, such as but not necessarily limited to the Aesculap AEOS™ Digital Microscope from Aesculap, Inc., forego use of eyepieces.

To enable the various software-based control aspects of the present disclosure, an electronic control unit (ECU) 30 may be placed in networked communication with the microscope 26 and the robot 25, with such two-way communication indicated by double-headed arrow CC₂₅ in FIG. 1. The ECU 30 may be configured to execute computer-readable code or instructions for performing one or more tasks involving use of the optical system 12. Although the ECU 30 is shown schematically as a unitary device for illustrative simplicity, the ECU 30 may include one or more networked computer devices, along with associated computer-readable media or memory, including a non-transitory (e.g., tangible) medium that participates in providing data/instructions that may be read by one or more processors (not shown).

The memory used for this purpose could take many forms, including but not limited to non-volatile media and volatile media. As will be appreciated, non-volatile media may include optical and/or magnetic disks and other persistent memory, while volatile media may include dynamic random-access memory (DRAM), static RAM (SRAM), etc., any or all which may constitute a main memory. Communication with the microscope 26 and the robot 25 may be achieved via a networked connection to input/output circuitry of the ECU 30. Other hardware not depicted but well established in the art may be included as part of the ECU 30, including but not limited to a local oscillator or high-speed clock, signal buffers, digital signal filters, etc. The ECU 30 could be enclosed within a moveable cabinet 35 or another suitable structure, e.g., a base 250 of the robot 25 that is mounted to or securely positioned on a floor 11 of the ophthalmic suite 10, to protect the ECU 30 from ingress of moisture or debris, and to cool the ECU 30, and to provide the necessary network and power connections. The ECU 30 could communicate with the display monitors 24 via display signals (CC₂₄) as part of the present strategy.

Referring briefly to FIGS. 2A and 2B, the constant downforce (CDF) assembly 18 as contemplated herein is configured to connect to the optical head 260 of the microscope 26 shown in FIG. 1 and described above. As appreciated in the art, various commercial options exist for connecting an external lens to the microscope 26 depending on its construction. In general, the CDF assembly 18 could be attached to the microscope 26 via either of the intervening connecting arms 40A or 40B of FIGS. 2A and 2B.

Referring first to FIG. 2A, the connecting arm 40A could include a connection ring 41 that is connected to the optical head 260. In this representative embodiment, e.g., the commercially-available RESIGHT® family from Carl Zeiss Meditec, Inc., oppositely-disposed tabs 42 disposed at a first end E1 of the connecting arm 40A are connected to or formed with the connection ring 41 are joined with the connecting arm 40A via revolute joints 44 to form a wishbone-shaped or flared arrangement as shown. Thus, the physician may rotate the connection ring 41 about an optical axis AA and swing the connecting arm 40A about a rotary axis RR of the revolute joints 44 to thereby locate first end E1 of the connecting arm 40A where desired within its available range of motion.

In this embodiment, a second end E2 of the connecting arm 40A may contain another revolute joint 144 or another application-suitable attachment mechanism. In accordance with the disclosure, the CDF assembly 18 is connected to the connecting arm 40A alone or in conjunction with a high-diopter lens (L1) 46. As appreciated in the art, such a lens 46 could be about 70-90 diopters, which the physician could selectively move into or out of alignment with the optical axis (AA) as needed. Thus, the lens 46 when used would be positioned between the optical head 260 and the CDF assembly 18.

A contact lens device 22 for its part is connectable to the CDF assembly 18 as shown. The contact lens device 22 includes the contact lens (L2) 22L noted above, which for its part is configured to be worn on a cornea 14C of the eye 140 when the patient is within the ophthalmic suite 10 of FIG. 1. As noted above, the CDF assembly 18 is configured to provide a constant downforce DF to the contact lens 22L, e.g., about 0.1 to 0.5 pounds per square inch gauge (psig) or another application-suitable downforce, and to self-level and thereby maintain the contact lens 22L in an approximately parallel orientation relative to the floor 11 of the ophthalmic suite 10. As used here in, “approximately parallel” means “within about ±5° to 10° of true parallel or within another application-suitable window of true parallel, while avoiding a true parallel orientation.

To ensure the desired orientation, a gimbal 36 could be connected to the distal end of the CDF assembly 18 and to the contact lens device 22. The gimbal 36 could be used to maintain the approximately parallel orientation of the contact lens 22L by limiting pitch and or roll of the contact lens device 22. Although shown schematically in FIG. 2A for illustrative simplicity, those skilled in the art will appreciate that commercially-available gimbals, e.g., for camera stability and robotic end-effector use, typically include an arrangement of rings connected at right angles to each other. As each constituent ring of the gimbal 36 can rotate independently of the other rings, the contact lens device 22 would likewise be able to rotate along multiple axes while maintaining the desired orientation relative to the floor 11 shown in FIG. 1.

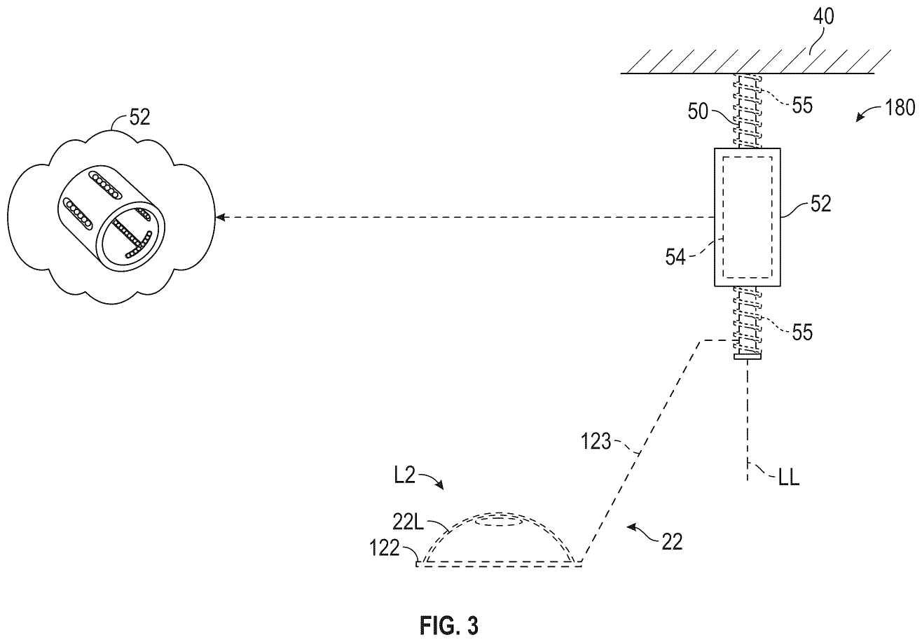

Referring to FIG. 2B, the alternatively constructed connecting arm 40B could be a vertically-translatable rod connected to an angled bracket 48 at end E1. The angled bracket 48 in turn could be connected to a main body 49, which in turn is connected to the above-described optical head 260 of FIG. 1 via a ring 141, e.g., as an OCULUS® BIOM® ready set. As appreciated in the art, a physician can swing the main body 49 and all connected components into and out of the optical axis AA, as indicated by double-headed arrow BB. The CDF assembly 18 as described below can therefore be used with different types of connecting arms, including but not limited to the connecting arms 40A and 40B of respective FIGS. 2A and 2B. Three possible configurations of the CDF assembly 18 will now be described with particular reference to FIGS. 3-5, with the connecting arms 40A and 40B of respective FIGS. 2A and 2B generically referenced as 40 in the remaining Figures.

Referring now to FIG. 3, the contact lens device 22 noted above includes the contact lens 22L, which is configured to be worn on the cornea 14C of the patient's eye 140 as shown in FIG. 2A. The contact lens 22L may be constructed of a rigid or semi-rigid gas permeable material, e.g., fluorosilicone acrylate, silicone acrylate, or another application-suitable material providing the required field of view. The contact lens device 22 is connectable to the distal end of a CDF assembly 180 in the illustrated embodiment of the CDF assembly 18 depicted in FIGS. 1-2B. For example, the contact lens device 22 could include a support frame 122 and a support frame arm 123. In such an embodiment, the support frame 122 is configured to support the contact lens 22L, e.g., around a perimeter or circumference thereof. The support frame arm 123 for its part may be welded to, integrally formed with, or otherwise connected to the support frame 122, and also connected to the CDF assembly 180, either removably or permanently in different implementations.

In the non-limiting embodiment of FIG. 3, the CDF assembly 180 is shown connected to the connecting arm 40, e.g., either of the connecting arms 40A or 40B of respective FIGS. 2A and 2B. The CDF assembly 180 in the illustrated construction includes a cylindrical rod or shaft 50 that is circumscribed by a bearing housing 52. The bearing housing 52, which contains instrument quality ball bearings 54 therein, is translatable along a longitudinal axis (LL) of the shaft 50. As appreciated in the art, linear ball bearings are used to minimize friction and ensure controlled, smooth linear motion in linear motion systems such as the illustrated bearing housing 52 and the contact lens device 22L connected thereto. Such motion can be caused by motion of the patient's eye 140 of FIG. 2A and/or the patient's head. Linear instrument bearings are commercially-available, e.g., the Thomson® family of linear bearings from Regal Rexnord Corporation of Belot, WI. The bearing housing 52 translates along the longitudinal axis (LL) of the shaft 50 via a ball track (not shown) within which the instrument ball bearings 54 are captive.

Vertical motion of the contact lens device 22L of FIG. 3 can be optimized in one or more embodiments using a suitable damping mechanism. For instance, in some implementations one or more constant force springs 55 could be connected to and/or surround the shaft 50. As appreciated in the art, constant force springs are configured to maintain a consistent force output in extension as well as in compression. In the present application, the constant force springs 55 would compress as the bearing housing 52 moves toward the optical head 260 of the microscope (see FIG. 1). When the patient moves back into a rest position, the compressed constant force springs 55 would slowly release their stored energy, thus precisely controlling the rate of descent of the contact lens device 22L.

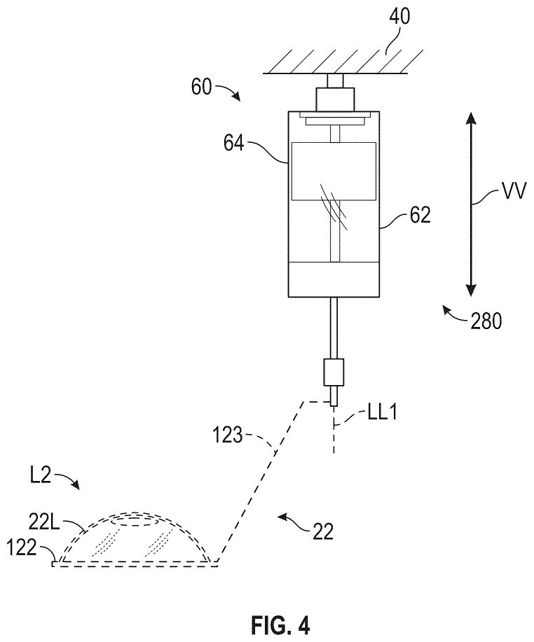

Referring now to FIG. 4, the desired constant downforce and self-leveling benefits of the contact-based WAVS 17 of FIG. 1 can be achieved in other ways within the scope of the disclosure. For example, a CDF assembly 280 could include a miniature gas spring 60 to provide precise downforce and control up/down motion (double-headed arrow VV). As understood in the art, miniature gas springs are designed to provide precise and controlled motion, often within a space-limited area. In general, the miniature gas spring 60 as contemplated herein may include a gas-filled cylinder 62 and a low-friction piston 64 that translates within the cylinder 62.

In the present application, such translation occurs in response to motion of the patient's eye 140 (FIG. 2A) or the patient's head (not shown). Changing pressure acting on the piston 64 results in smooth, near-frictionless linear motion. The gas spring 60 may be configured to slow motion of the piston 64 when a force is imparted by the patient's eye 140 and/or patient's head. Use of added resistance and damping of motion of the contact lens device 22 and the connected wide-angle contact lens (L2) 22L may be implemented for optimal low-friction performance.

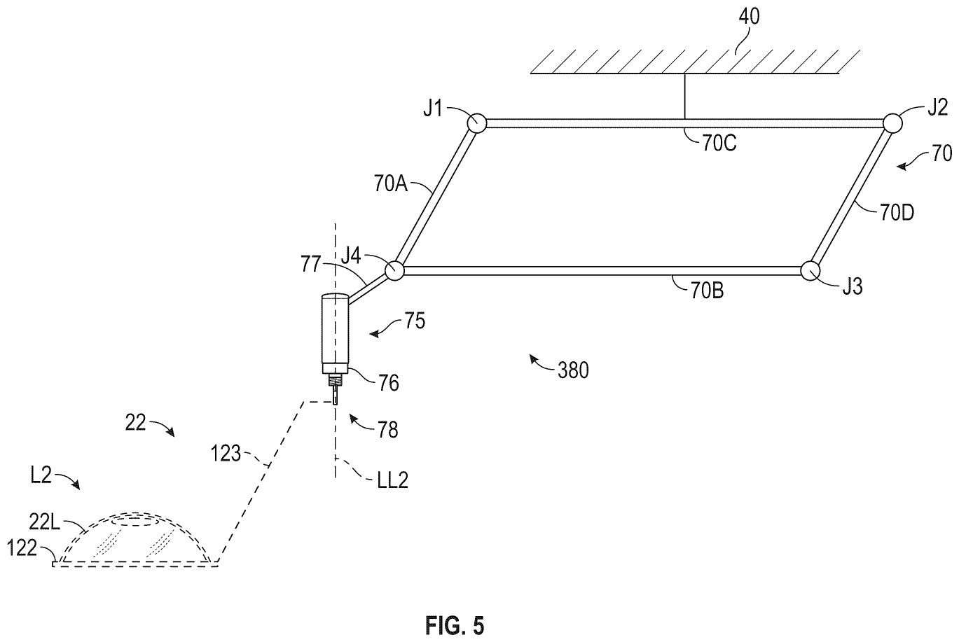

Referring to FIG. 5, the contact-based WAVS 17 of FIG. 1 in yet another possible embodiment may include a CDF assembly 380 equipped with a four-bar mechanism 70. The four-bar mechanism 70 as contemplated herein and as understood in the art includes first, second, third, and fourth linkages or bars 70A, 70B, 70C, 70D interconnected by revolute joints J1, J2, J3, and J4 as shown. This arrangement thus provides up/down motion and hold the contact lens device 22 approximately parallel to the floor 11 of FIG. 1, e.g., within a slight tolerance of true parallel as permitted by the gimbal 36 of FIG. 2A or other suitable structure.

In one or more embodiments, the four-bar mechanism 70 could be operatively connected to a low-friction air cylinder 75 fed by regulated pneumatic pressure (not shown) to maintain the constant downforce, e.g., about 0.1 to 0.5 psig or another patient and application-suitable constant downforce. The low-friction air cylinder 75 in turn has a low-friction piston 76 disposed therewithin. A longitudinal axis (LL2) of the piston 76 could be laterally offset from the four-bar mechanism 70 as shown, e.g., via an interconnecting piece 77 connected to the low-friction air cylinder 75. The contact lens device 22 would then be connected to an end 78 of the piston 76 of the low-friction air cylinder, 75 such that the air cylinder 75 would provide the above-noted constant downforce on the contact lens (L2) 22L. At the same time, the optional four-bar mechanism 70 maintains the desired approximately parallel orientation relative to the floor 11 of FIG. 1—without permitting a true parallel orientation-along with upward or downward motion of the contact lens device 22 in response to imparted motion of the patient. The low-friction air cylinder 75 thus counterbalances the vertically-guided load, which in this case includes the contact lens device 22, its support frame 122, and its support arm 123.

Low-friction air cylinders, e.g., the commercially-available Airpel-AB® air cylinders from Airpot® Corporation of Norwalk, CT, are constructed to provide smooth and efficient, and essentially frictionless linear motion, and thus are usable within the scope of the present disclosure. In terms of minimal friction, this can be achieved by constructing the cylinder 75 from materials having a low coefficient of friction, e.g., borosilicate glass, along with process steps such as machining. Used in conjunction with the illustrated four-bar mechanism, the low-friction air cylinder 75 and the low-friction piston 76 disposed therewithin applies the desired constant downforce smoothly and efficiently.

As will be appreciated by those skilled in the art in view of the foregoing disclosure, the solutions presented above ensure a constant downforce on the contact lens 22L in a precise manner that ensures the cornea is not distorted. This occurs without admitting air bubbles behind the contact lens 22L. The contact lens 22L is maintained in a substantially parallel (but not true parallel) orientation relative to the floor 11 of the ophthalmic suite of FIG. 1, as opposed to a plane of the iris. The slight non-parallel tolerance enabled, e.g., by the gimbal of FIG. 2A, ensures this orientation. The patient's head can then move up or down with respiration, with the lowest possible friction along the axis of such vertical movement, both in terms of static friction (“stiction ”) and Coulomb friction. Embodiments described above could be either autoclavable or disposable in different configurations, thereby facilitating more widespread adoption of the present teachings. These and other attendant benefits will be readily appreciated by those skilled in the art in view of the foregoing disclosure.

The detailed description and the drawings are intended to be supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings or the characteristics of

various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

What is claimed is:

1. A contact-based wide-angle visualization system (“WAVS”) for use in an ophthalmic suite having a microscope, the contact-based WAVS comprising:
a constant downforce (CDF) assembly having a proximal end and a distal end, wherein the proximal end of the CDF assembly is configured to connect to an optical head of the microscope via an intervening connecting arm; and
a contact lens device that is connectable to the distal end of the CDF assembly, and that includes a contact lens configured to be worn on a cornea of a patient's eye in the ophthalmic suite, wherein the CDF assembly is configured to (i) provide a constant downforce to the contact lens device, and (ii) self-level and thereby maintain the contact lens device in an approximately parallel orientation relative to a floor of the ophthalmic suite.

2. The contact-based WAVS of claim 1, further comprising:
a gimbal connected to the distal end of the CDF assembly and to contact lens device, wherein the gimbal maintains the approximately parallel orientation by limiting a pitch and/or a roll of the contact lens device.

3. The contact-based WAVS of claim 1, wherein the contact lens device includes:
a support frame configured to support the contact lens; and
a support frame arm connected to the support frame and to the CDF assembly.

4. The contact-based WAVS of claim 1, wherein the CDF assembly includes a shaft that is circumscribed by a bearing housing containing instrument bearings therein, and wherein the bearing housing is translatable along a longitudinal axis of the shaft.

5. The contact-based WAVS of claim 4, further comprising:
one or more constant force springs connected to or surrounding the shaft.

6. The contact-based WAVS of claim 1, wherein the CDF assembly includes a miniature gas spring.

7. The contact-based WAVS of claim 1, wherein the CDF assembly includes a four-bar mechanism operatively connected to a low-friction air cylinder, the low-friction air cylinder having a low-friction piston disposed therewithin, and wherein a longitudinal axis of the piston is laterally offset from the four-bar mechanism.

8. The contact-based WAVS of claim 7, wherein an end of the piston is operatively connected to the contact lens device.

9. The contact-based WAVS of claim 8, wherein the low-friction air cylinder is constructed from borosilicate glass.

10. The contact-based WAVS of claim 1, further comprising:
the intervening connecting arm.

11. A contact-based wide-angle visualization system (“WAVS”) for use in an ophthalmic suite having a microscope, the contact-based WAVS comprising:
a constant downforce (CDF) assembly having a proximal end and a distal end, wherein the proximal end of the CDF assembly is configured to connect to an optical head of the microscope via an intervening connecting arm;
a contact lens device that is connectable to the distal end of the CDF assembly and that includes a contact lens configured to be worn on a cornea of a patient's eye in the ophthalmic suite, a support frame configured to support the contact lens, and a support frame arm connected to the support frame and to the CDF assembly; and
a gimbal connected to the distal end of the CDF assembly and to contact lens device, wherein the CDF assembly is configured to provide a constant downforce to the contact lens device, and to self-level and thereby maintain the contact lens device in an approximately parallel orientation relative to a floor of the ophthalmic suite, and wherein the gimbal maintains the approximately parallel orientation by limiting a pitch and/or a roll of the contact lens device.

12. The contact-based WAVS of claim 11, wherein the CDF assembly includes a shaft that is circumscribed by a bearing housing containing instrument bearings therein, and wherein the bearing housing is translatable along a longitudinal axis of the shaft.

13. The contact-based WAVS of claim 12, further comprising:
one or more constant force springs connected to or surrounding the shaft.

14. The contact-based WAVS of claim 11, wherein the CDF assembly includes a miniature gas spring.

15. The contact-based WAVS of claim 11, wherein the CDF assembly includes a four-bar mechanism operatively connected to a low-friction air cylinder.

16. The contact-based WAVS of claim 15, wherein the low-friction air cylinder has a piston disposed therewithin, a longitudinal axis of the piston is laterally offset from the four-bar mechanism, and an end of the piston is operatively connected to the contact lens device.

17. A system comprising:
a connecting arm configured to connect to an optical head of a microscope; and
a contact-based wide-angle visualization system (“WAVS”) for use in an ophthalmic suite having a microscope, the contact-based WAVS comprising:
a constant downforce (CDF) assembly having a proximal end and a distal end, wherein the proximal end of the CDF assembly is configured to connect to the connecting arm, wherein the CDF assembly includes a shaft that is circumscribed by a bearing housing containing instrument bearings therein, the bearing housing being translatable along a longitudinal axis of the shaft, and wherein one or more constant force springs connected to or surrounding the shaft; and
a contact lens device that is connectable to the distal end of the CDF assembly and that includes a contact lens configured to be worn on a cornea of a patient's eye in the ophthalmic suite, wherein the CDF assembly is configured to provide a constant downforce to the contact lens device, and to self-level and thereby maintain the contact lens device in an approximately parallel orientation relative to a floor of the ophthalmic suite.

18. The system of claim 17, further comprising:
a gimbal connected to the distal end of the CDF assembly and to contact lens device, wherein the gimbal maintains the approximately parallel orientation by limiting a pitch and/or a roll of the contact lens device.

19. The system of claim 17, wherein the contact lens device includes:
a support frame configured to support the contact lens.

20. The system of claim 19, wherein the contact lens device includes:
a support frame arm that is connected to the support frame and to the CDF assembly.
